(19) United States
(12) Patent Application Publication
(10) Pub. No.: US 2016/0038061 A1
(43) Pub. Date: Feb. 11, 2016

(54) METHOD FOR DETECTING FALLS AND A FALL DETECTOR

(71) Applicant: KONINKLIJKE PHILIPS N.V., EINDHOVEN (NL)

(72) Inventors: PATRICK KECHICHIAN, EINDHOVEN (NL); WEI ZHANG, EINDHOVEN (NL)

(21) Appl. No.: 14/779,187

(22) PCT Filed: Feb. 21, 2014
(86) PCT No.: PCT/IB2014/059146

§ 371 Date: Sep. 22, 2015

Related U.S. Application Data
(60) Provisional application No. 61/804,222, filed on Mar. 22, 2013.

(51) Int. Cl. A61B 5/11; A61B 5/0488; A61B 5/01; A61B 5/00; A61B 5/024

(52) U.S. Cl. CPC A61B 5/1117; A61B 5/4035; A61B 5/024; A61B 5/01; A61B 5/441; A61B 5/0488; A61B 5/7221

(57) ABSTRACT

There is provided a method of detecting a fall by a user, the method comprising detecting whether a user has potentially experienced a fall event from measurements of the movements of the user; on detecting a potential fall event, determining the activity level of the user and a measure of a autonomic nervous system, ANS, response for the user associated with the potential fall event; comparing the determined activity level and the measure of the ANS response to a user profile relating activity level and ANS response for the user; and determining whether the potential fall event is a fall based on the result of the comparison.

### TECHNICAL FIELD OF THE INVENTION

The invention relates to a method for detecting falls by a user and a fall detector implementing the same, and in particular relates to a method for detecting falls and a fall detector that provides increased fall detection reliability.

### BACKGROUND TO THE INVENTION

Falls affect millions of people each year and result in significant injuries, particularly among the elderly. In fact, it has been estimated that falls are one of the top three causes of death in elderly people. A fall is defined as a sudden, uncontrolled and unintentional downward displacement of the body to the ground, followed by an impact, after which the body stays down on the ground.

Personal Help Buttons (PHBs) are available that require the user to push the button to summon help in an emergency. However, if the user suffers a severe fall (for example if they are knocked unconscious), the user might be unable to push the button, which might mean that help doesn't arrive for a significant period of time, particularly if the user lives alone.

Fall detectors are also available that process the output of one or more movement sensors to determine if the user has suffered a fall. Most existing body-worn fall detectors make use of an accelerometer (usually an accelerometer that measures acceleration in three dimensions) and they try to infer the occurrence of a fall by processing the time series generated by the accelerometer. Some fall detectors can also include an air pressure sensor, for example as described in WO 2004/114245. On detecting a fall, an alarm is triggered by the fall detector.

Some fall detectors are designed to be worn as a pendant around the neck of the user, whereas others are designed to be worn on the torso or limbs of the user, for example at the wrist. However, the wrist is capable of complex movement patterns and has a large range of movement, which means that existing fall detection methods based on analysing measurements from an accelerometer do not provide a sufficiently high detection rate while minimising the number of false alarms for this type of fall detector.

The paper “A new approach to improve the fall detection in elderly: monitoring of the autonomic nervous system activation” by R. Nocua, N. Noury, C. Gehin, A. Dittmar and E. McAdams; WC 2009, IFMBE Proceedings 25/VII, pp. 681-684, 2009 suggests that falls produce a measurable response in the autonomic nervous system of the user, and that measurements of skin resistance and heart rate can be used to classify whether the user has fallen.

### SUMMARY OF THE INVENTION

However, it has been found that as the response of physiological characteristics such as heart rate and skin resistance to stress or increases in physical activity levels is highly user-specific, the assessment of an autonomic nervous system response to a fall using measurements of heart rate and skin resistance still does not provide an acceptable false alarm rate.

Therefore, there is a need for a way of detecting falls that makes use of measurements that can indicate an autonomic nervous system response (and sometimes specifically a sympathetic nervous system response), and that provides a reduced false alarm rate.

Therefore, according to a first aspect of the invention, there is provided a method of detecting a fall by a user, the method comprising detecting whether a user has potentially experienced a fall event from measurements of the movements of the user; on detecting a potential fall event, determining the activity level of the user and a measure of an autonomic nervous system, ANS, response for the user associated with the potential fall event; comparing the determined activity level and the measure of the ANS response to a user profile relating activity level and ANS response for the user; and determining whether the potential fall event is a fall based on the result of the comparison.

In some embodiments, the step of comparing the determined activity level and the measure of the ANS response to a user profile comprises using the profile to determine the likelihood of the user having the determined activity level and measure of ANS response, and wherein the step of determining whether the potential fall event is a fall uses the determined likelihood.

In some embodiments, the user profile relates typical activity levels and typical ANS responses for the user, and wherein the step of determining whether the potential fall event is a fall comprises determining that the potential fall event is a fall if the determined likelihood is below a threshold, and determining that the potential fall event is not a fall if the determined likelihood is above a threshold.

In some embodiments, in the event that it is determined that the potential fall event is a fall and an indication is subsequently received that the fall event was not a fall, the method further comprises the step of adjusting the value of the threshold. The indication that the fall event was not a fall may be an input to the fall detector by the user or a signal received from a remote computer associated with the fall detector.

The user profile can be a joint probability distribution of activity level and ANS response.

The method can further comprise the step of determining the user profile relating activity level and ANS response for the user by: (i) obtaining pairs of measurements of the activity level and ANS response for the user for a plurality of time periods; and (ii) determining a joint distribution of activity level and ANS response for the user from the obtained pairs of measurements.

The step of determining the user profile relating activity level and ANS response for the user may comprise determining a plurality of user profiles relating activity level and ANS response for the user, wherein each profile relates the activity level and ANS response for a particular time period during the day.

The step of determining the user profile relating activity level and ANS response for the user may comprise the step of obtaining pairs of measurements of the activity level and ANS response of the user depending on the measured activity level of the user.

The step of obtaining pairs of measurements of the activity level and ANS response for the user may comprise discarding any pair of measurements obtained for a time period in which a potential fall by the user is detected.

The step of obtaining pairs of measurements of the activity level and ANS response for the user may also comprise discarding any pair of measurements obtained where the measured ANS responses do not correspond to a sympathetic nervous system, SNS, response.

The joint distribution can be a joint probability density function or a joint probability mass function.

In some embodiments, the step of determining the activity level and a measure of an ANS response comprises determining the activity level and/or the measure of ANS response from the measurements of the movements of the user.

In other embodiments, the step of determining the activity level and a measure of the ANS response comprises determining the activity level from the measurements of the movements of the user and the measure of ANS response from measurements of a physiological characteristic of the user by a physiological characteristic sensor.

Preferably, the measure of ANS response is one or more of skin temperature, skin conductance, heart rate and any other heart-related characteristic of the user.

Furthermore, the ANS response can be categorized as an SNS response according to whether an increase in heart-rate, constriction of blood vessels, and increase and/or activation of sweat secretion is also measured.

In preferred embodiments, the step of detecting whether a user has potentially experienced a fall event comprises measuring the movements of the user; and analysing the measurements of the movements of the user to identify one or more characteristics associated with a fall.

Preferably, the one or more characteristics associated with a fall are selected from: (i) a height change, (ii) an impact, (iii) a free-fall, (iv) a change in orientation from upright to horizontal, and (v) a period of inactivity.

According to a second aspect of the invention, there is provided a computer program product comprising computer readable code embodied therein, the computer readable code being configured such that, upon execution by a suitable computer or processor, the computer or processor performs the method as described above.

According to a third aspect of the invention, there is provided a fall detector for detecting falls by a user, the fall detector comprising a movement sensor for measuring the movements of the user; and a processor configured to detect whether the user has potentially experienced a fall event from measurements of the movements of the user from the sensor; on detecting a potential fall event, determine the activity level of the user and a measure of an autonomic nervous system, ANS, response for the user associated with the potential fall event; compare the determined activity level and the measure of the ANS response to a profile relating activity level and ANS response for the user; and determine whether the potential fall event is a fall based on the result of the comparison.

In some embodiments, the processor is configured to compare the determined activity level and the measure of the ANS response to a user profile to determine the likelihood of the user having the determined activity level and measure of ANS response, and to use the determined likelihood to determine whether the potential fall event is a fall.

In some embodiments where the user profile relates typical activity levels and typical ANS responses for the user, the processor is configured to determine that the potential fall event is a fall if the determined likelihood is below a threshold, and to determine that the potential fall event is not a fall if the determined likelihood is above a threshold.

In some embodiments the processor is further configured to determine the user profile relating activity level and ANS response for the user by (i) determining pairs of measurements of the activity level and ANS response for the user for a plurality of time periods; and (ii) determining a joint distribution of activity level and ANS response for the user from the obtained pairs of measurements.

In some embodiments the processor is further configured to determine a plurality of user profiles relating activity level and ANS response for the user, wherein each profile relates the activity level and ANS response for a particular time period during the day.

The processor can be configured to discard pairs of measurements of the activity level and ANS response for the user that are obtained during a time period in which the processor detects a potential fall by the user.

In some embodiments, the processor is configured to determine the activity level and a measure of an ANS response from the measurements of the movements of the user.

In alternative embodiments, the fall detector further comprises a physiological characteristic sensor for measuring a physiological characteristic of the user; and wherein the processor is configured to determine the activity level from the measurements of the movements of the user and to determine the measure of ANS response from the measurements of the physiological characteristic by the physiological characteristic sensor.

Preferably the measure of ANS response is one or more of skin temperature, skin conductance, heart rate and any other heart-related characteristic of the user.

Preferably the processor is configured to detect whether a user has potentially experienced a fall event by analysing the measurements of the movements of the user to identify one or more characteristics associated with a fall.

Preferably the one or more characteristics associated with a fall are selected from: (i) a height change, (ii) an impact, (iii) a free-fall, (iv) a change in orientation from upright to horizontal, and (v) a period of inactivity.

### BRIEF DESCRIPTION OF THE DRAWINGS

Exemplary embodiments of the invention will now be described, by way of example only, with reference to the following drawings, in which:

FIG. 1 is a block diagram of a fall detector in accordance with the invention;

FIG. 2 is a graph illustrating an exemplary signal from a photoplethysmograph sensor;

FIG. 3 is a flow chart illustrating a method of generating a user conditioning profile;

FIG. 4 is a flow chart illustrating a method of detecting falls in accordance with an embodiment of the invention; and

FIG. 5 is a contour plot of an exemplary joint distribution of heart rate and activity level.

### DETAILED DESCRIPTION OF THE PREFERRED EMBODIMENTS

As described above in the Background section, it has been found that existing fall detection methods based on analysing measurements from movement sensors in a wrist-worn fall detector do not provide a sufficiently high detection rate while limiting the number of false alarms due to the movements of the wrist that occur in, what is referred to herein as, ‘activities of daily living’, ADL, which are the typical movements of the user's wrist that occur during daily activities such as walking, sitting, etc. that are not connected with falls by the user.

It is known that when a person accidentally falls, the process of falling, from losing balance to the impact with the ground and the (possible) inability to get up after the fall, causes a response in the ANS of the user, which can be measured as a change in the skin conductivity, skin temperature and/or heart rate of the user as a result of the stress suffered. However, a movement, such as sitting down or intentionally ‘falling’ onto a chair, which might appear to be a fall from an analysis of movement sensor measurements alone, would not cause this stress response.

Thus, the use of measurements of physiological characteristics such as heart rate and skin conductivity as indicators of an autonomic nervous system response can help to filter out some of these false fall detections since significant changes in, e.g. heart rate, can often occur around a fall event, either due to the ‘flight-or-fight’ response of the body during a fall or due to the resulting trauma.

However, heart rate (for example), is a highly user-dependent measure and rest and elevated heart rate levels can depend on a number of factors such as a person's health, fitness level, and age, for example. Users that have better conditioning (e.g. that are fitter) tend to have lower heart rates during high levels of physical activity than users with poorer conditioning who have higher heart rates for similar activities.

Therefore, the invention provides an improved technique for using measurements of physiological characteristics indicative of an autonomic nervous system (ANS) response to detect falls. In particular, the invention provides that a profile for the user that represents the general condition of the user is established that relates daily activity levels to a measure (or multiple measures) of ANS response, and then, when a potential fall event is detected (for example a free-fall followed by an impact is detected), the current activity level and measure(s) of the ANS response are compared to the profile in order to determine whether the event is a fall or whether it is an ‘ADL’, i.e. a non-fall.

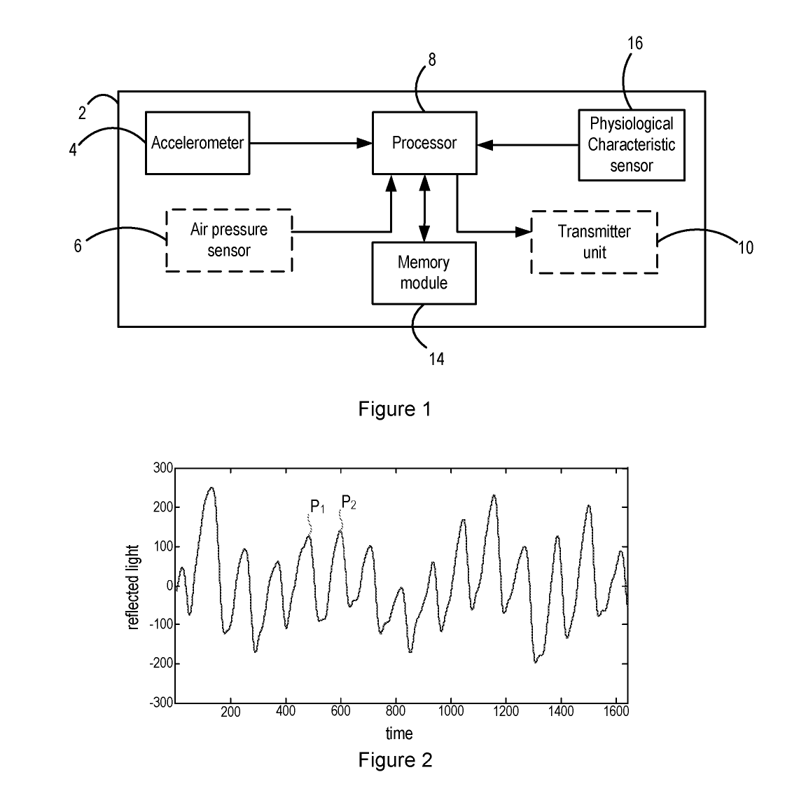

A fall detector 2 according to an embodiment of the invention is shown in FIG. 1. In a preferred embodiment of the invention, the fall detector 2 is designed to be worn by a user on their wrist, although it will be appreciated that the invention is not limited to this use, and the fall detector 2 could instead be designed to be worn at the user's waist, on their chest or back, as a pendant around their neck or carried in their pocket.

In this exemplary embodiment, the fall detector 2 comprises two movement sensors—an accelerometer 4 and pressure sensor 6—which are connected to a processor 8. The processor 8 receives measurements from the movement sensors 4, 6, and processes the measurements to determine if a user of the fall detector 2 may have suffered a fall. Although two movement sensors are shown in this embodiment, it will be appreciated that fall detectors according to alternative embodiments may comprise only one movement sensor (for example just the accelerometer 4 with the pressure sensor 6 being omitted). In yet further embodiments, the fall detector 2 can comprise a gyroscope and/or electromyography (EMG) sensor(s) in addition or alternatively to the pressure sensor 6.

The fall detector 2 also comprises a transmitter unit 10 that allows the fall detector 2 to transmit an alarm signal to a base station associated with the fall detector 2 (which can then issue an alarm or summon help from a healthcare provider or the emergency services) or directly to a remote station (for example located in call centre of a healthcare provider) if a fall is detected, so that assistance can be summoned for the user. In some embodiments, the processor 8 in the fall detector 2 may not execute an algorithm on the data from the sensors 4, 6 to determine if the user may have fallen; instead the processor 8 and transmitter unit 10 may provide the raw data from the sensors 4, 6 to the base station and a processor in the base station can execute the algorithm on the data from the sensors 4, 6 to determine if the user may have fallen.

The fall detector 2 also comprises a memory module 14 that is connected to the processor 8 and that can store measurement data from the movement sensors 4, 6, computer readable code for use by the processor 8 and/or data for a user conditioning profile, which is described in more detail below.

It will be appreciated that the memory module 14 may only store the latest measurement data and that measurement data may also be transmitted using transmitter unit 10 to a remote server on or via a base station for storage.

The fall detector 2 further comprises a sensor or sensors 16 for measuring one or more physiological characteristics of the user. The physiological characteristics can comprise any of the skin temperature, skin conductance, heart rate, other heart-related characteristics or any other physiological characteristic that can indicate a response by the autonomic nervous system of the user to an event.

In a wrist-worn fall detector 2 where the sensor 16 measures skin conductivity, the sensor 16 is preferably arranged to contact the skin of the user on the volar side of their wrist. In some embodiments, the fall detector 2 comprises multiple skin conductivity sensors 16 that are to be placed at different positions on the user's body. In this case, at least one of those skin conductivity sensors 16 can be integrated into a separate housing to the rest of the components of the fall detector 2.

As appreciated by those skilled in the art, suitable sensors 16 for measuring the heart rate and other heart-related characteristics include an electrocardiography (ECG) device or a photoplethysmography (PPG) device.

While ECGs measure the electrical activity of the heart, PPGs measure changes in the blood volume which is correlated to the beat-to-beat intervals of the heart, and can also be used to measure the oxygen saturation of the blood. Another useful feature of the signal produced by a PPG sensor which is related to or is indicative of the ANS response is the amplitude of the resulting pulse which is related to the distensibility of the arteries and is an indicator of sympathetic tone.

It will be appreciated by those skilled in the art that, in some embodiments, for example where the physiological characteristic to be measured is the heart rate and the fall detector 2 is attached to the user's wrist, the pulsing of blood through the arteries in the user's arm as the heart beats may be detectable in the signal from the accelerometer 4, in which case the signal can be processed to extract the heart rate of the user and a separate physiological characteristic sensor 16 is not required.

In the following, an embodiment of the invention that comprises a PPG sensor 16 will be presented since the signal displays useful features of the ANS response of the user and is also easy to implement in a wearable device 2. As is known, a PPG sensor 16 typically consists of a light source, e.g. a LED, which transmits light of a certain wavelength, e.g. 940 nm, into human tissue, and a light-sensitive sensor such as a photo-diode which reacts to the transmitted or reflected light. Where both sensors are applied to the same area of tissue, the reflected light is measured. If the light sensor is placed at another part of the body, e.g. opposite the light source such as on the tip of a finger, then the transmitted light is measured. An increase in blood volume increases the amount of light reflected while decreasing the amount of light transmitted, and thus the two configurations produce waveforms with an inverse amplitude relationship.

An exemplary filtered (DC-removed) PPG waveform is shown in FIG. 2, which indicates the amount of reflected light measured at the wrist. The distance between two consecutive peaks (e.g. P₁ and P₂) corresponds to the peak-to-peak interval, the inverse of which is the heart-rate, denoted r(n) herein, measured in beats-per-minute (bpm).

Although not shown in the embodiment of FIG. 1, the fall detector 2 may further comprise an audible alarm unit that can be activated by the processor 8 in the event that the processor 8 determines that the user has suffered a fall. The fall detector 2 may also be provided with a button (also not shown in FIG. 1) that allows the user to manually activate the audible alarm unit if they require assistance (or deactivate the alarm if assistance is not required).

In the illustrated embodiment, all of the components of the fall detector 2 are integrated into a single housing that is to be placed in contact with the user's skin. In alternative embodiments, for example where part of the fall detector is in the form of a pendant to be worn around the user's neck (and so might not be in contact with the user's skin at all times), the physiological characteristic sensor 16 can be provided in a housing that is separate from the pendant (the pendant including the movement sensor(s) (e.g. accelerometer 4 and pressure sensor 6)), so that the physiological characteristic sensor 16 can be in contact with the user's skin during use.

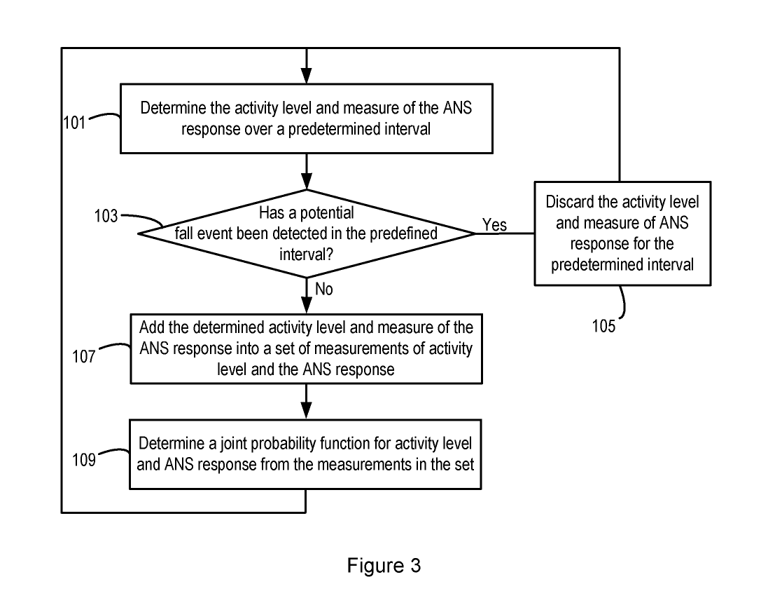

As described above, as part of determining whether the user of the fall detector 2 has fallen, a profile for the user is required that relates daily activity levels to a measure (or multiple measures) of ANS response. FIG. 3 illustrates a method of generating a user conditioning profile. In this illustrated embodiment, the physiological characteristic used as the measure of ANS response is the user's heart rate, which is denoted r(n).

In a first step, step 101, the activity level and the measure of the ANS response of the user (i.e. heart rate in this embodiment) are measured over a predefined interval. Where the interval is defined as a period of time for which a data sample of the activity level and ANS response measure is calculated and available, the length of the predefined interval can range from 30 seconds to 1 minute. However, it is important to note that to collect a sufficiently large number of data points a period P on the order of 5-10 minutes is defined where P contains the contiguous or overlapping intervals.

The activity level is a measure of the level and/or type of activity (e.g. motion) of the user over the interval and can be determined in a number of ways. In preferred embodiments, the activity level is determined by the processor 8 from the signals from the movement sensors 4, 6 in the fall detector 2. In other embodiments, a separate device can be provided for the user to wear or carry that includes one or more movement sensors, such as an accelerometer, gyroscope, etc.

Where the activity level is determined from accelerometer measurements, the signal from the accelerometer 4 represents acceleration values along three orthogonal axes which can be sampled at regular intervals. The resulting signals be denoted by x(n), y(n), and z(n), where n is the discrete-time index. The sampling frequency may be set, for example, to between 50 and 150 Hz.

In one simple embodiment, the instantaneous activity level, a(n) can be given by the magnitude of the acceleration vector, i.e.

α(n)=\sqrt{x²(n)+y²(n)+z²(n)} (1)

The activity level for the predefined interval can be the average of a plurality of instantaneous activity level measurements obtained over the interval. In this case, the activity level for the predefined interval is denoted α̅ᵢ, where 1≦i≦N and N is the number of predefined intervals each of length T acceleration samples. The heart-rate can similarly be sampled a number of times over the predefined interval and averaged to give the heart rate for the predefined interval as r̅ᵢ. In some embodiments, α̅ᵢ and r̅ᵢ are given by

$$\begin{matrix}{\overline{α}}_{i} = \frac{1}{T} {\sum }_{m = 1}^{T} {α}_{i} ( m ) & ( 2 ) \\ {\overline{r}}_{i} = \frac{1}{T} {\sum }_{m = 1}^{T} {r}_{i} ( m ) & ( 3 )\end{matrix}$$

where m is the sample index within the interval i and the values used to form an activity level-heart rate pair ( α̅ᵢ, r̅ᵢ) for the predefined interval (1≦m≦T). It will be appreciated that the heart-rate measure might be sampled at a different sampling frequency to the acceleration, and thus is based on a different number of samples over the same time interval.

Another measure of the heart-rate during a pre-defined interval might be the average rate of change in heart-rate which can be defined as

$${\overline{r}}_{i} = \frac{1}{T - 1} {\sum }_{m = 2}^{T} [ {r}_{i} ( m ) - {r}_{i} ( m - 1 ) ] \qquad ( 4 )$$

This measure can also indicate whether the ANS response measured over the T samples corresponds to a SNS response, i.e. r̅ᵢ>0.

Besides an instantaneous activity level based on the norm of the raw 3D acceleration signal, a more robust computation based on the integration of the norm over a certain time period (for example 1 second) can be used. As above, an average of the values for the plurality of time periods across the interval can be used as the measure of the activity level and ANS response.

Other measures of activity level can be obtained by further processing the norm of the raw 3D acceleration signal (i.e. according to equation (1)) with a low-pass filter, a median filter or a moving-average filter to provide a more robust estimation of activity level.

Alternatively or in addition to the measures of activity level provided above, the processor 8 can process the signals from the movement sensors 4, 6 to determine the posture of the user during the activity (for example, standing up or lying down), in which case the value of α(n) is given by the output of a posture discrimination algorithm and takes on binary values, i.e. α(n)=(0,1) where 0 can correspond to lying down and 1 to standing. In other embodiments, more complex posture/activity discrimination algorithms can be used which are able to categorise multiple types of activity, for example walking, running, sitting down, etc., in which case a(n) can take on multiple discrete values. Suitable algorithms for determining these postures and/or activities from movement sensor signals will be known to those skilled in the art and will not be described in further detail herein.

The measure of activity level used by the fall detector 2 can be selected in view of the trade-off of robustness against computation complexity and power-consumption.

In order for the user conditioning profile to be useful for assisting in classifying potential fall events as falls or dismissing fall-like events as normal activities for the user, the user conditioning profile generated according to the method in FIG. 3 should be generated from activity level and heart rate measurements that are collected when the user is performing their normal daily activities. Thus, once an activity level-heart rate pair (α(n),r(n)) or ( α̅ᵢ, r̅ᵢ) has been determined for the predefined interval in step 101, it is determined in step 103 whether a potential fall event has been detected during the predefined interval. The detection of a potential fall event is described in more detail in connection with step 203 below.

If a fall event has been detected in the predefined interval for which activity level-heart rate pair (α(n),r(n)) or ( α̅ᵢ, r̅ᵢ) has been determined, the pair is discarded (step 105) and the measurements are not used in generating the user conditioning profile. The method thus returns to step 101 in which the activity level and measure of the ANS response is determined for the next predefined interval. The data could be collected during periods of length P a number of times a day (e.g., every 2 hours for 5-10 minutes), but the frequency with which data is collected could also depend on the power budget for the fall detector 2.

In some embodiments, the data collection period of length P can be triggered depending on the activity level of the user so that a more representative profile of the user is maintained. For example, if the system has collected sufficient activity level-heart rate pairs (α(n),r(n)) or ( α̅ᵢ, r̅ᵢ) during periods when the user's activity level has been low, then it will wait for periods when the user's activity level is higher to begin collecting data and update the user's profile. These embodiments provide the advantage that the user profile will not be updated when the user is asleep or inactive.

If no fall event has been detected for the predefined interval, the method moves to step 107 in which the activity level and heart rate pair (α(n),r(n)) or ( α̅ᵢ, r̅ᵢ) are added to a set of previously collected activity level and heart rate pairs (i.e. pairs collected during previous predefined intervals).

Then, in step 109, a joint probability function (which represents the user conditioning profile) for activity level and ANS response (heart rate) is determined from the measurements in the set.

In some embodiments, the joint probability function is a joint probability density function (pdf), denoted p(α,r), which can be estimated from the activity level-heart rate pairs in the set.

An exemplary technique for determining the joint probability function uses a Gaussian Mixture Model containing M mixtures, and is given by

$$p ( α , r \vert Θ ) = {\sum }_{i = 1}^{M} {β}_{i} {p}_{i} ( α , r \vert {θ}_{i} ) \qquad ( 5 )$$

where βi represents the probability of drawing a and r from mixture element i, i.e.

$${\sum }_{i = 1}^{M} {β}_{i} = 1 \\ and Θ = \{ θ1 , θ2 , … , θ M \} . \qquad ( 6 )$$

The parameters θi describe the underlying Gaussian density functions which are characterized by their mean and covariance, i.e. θi={μi, Σi} where μi is a 2-dimensional mean vector and Σi is the 2-by-2 covariance matrix of mixture element i, resulting in

$${p}_{i} ( x \vert Θ ) = \frac{1}{2 π {\vert {Σ}_{i} \vert}^{1 / 2}} {e}^{- \frac{1}{2} {( x - {μ}_{i} )}^{T} {Σ}_{i}^{- 1} ( x - {μ}_{i} )} \\ where x = {[ α , r ]}^{T} . \qquad ( 7 )$$

The parameters Θ and βᵢ that increase the maximum likelihood of the mixture model over the observed activity level and heart-rate values can be estimated using an algorithm such as the expectation-maximization (EM) algorithm, which is known to those skilled in the art.

In one embodiment, the mixture model is updated by first storing all the observed values of α and r, and then the probability density function is determined using the EM algorithm.

In another embodiment, the values are not stored and an (e.g. online) version of a probability density function estimation algorithm is used to update p(α, r|Θ).

It will be appreciated that other algorithms and models can be used for estimation of the probability density function, and such algorithms and models will be known to those skilled in the art.

In further embodiments, the M elements of the mixture can be allocated to different parts of the day and updated independently. This embodiment is useful as the typical activity levels of a user may vary throughout the day (for example they may regularly go for a walk in the morning while being less active in the afternoon), and thus there may be a respective function (profile) for different parts of the day.

In a further embodiment, in which there may be a constraint on memory size, the probability density functions are updated by first discarding the X oldest (α, r) data points before updating with X new (α, r) data points. In some implementations, this resampling may not be based on the time-stamp of the points, but is instead based on replacing the nearest neighbour currently in the data set.

In yet another embodiment of the invention where the heart-rate measurement corresponds to the average heart-rate per interval, the resulting heart-rate values can be quantized to the nearest fifth beat per minute (e.g., 87 becomes 85), and a separate probability density function over the activity level estimated for each quantized value.

In practice, since the probability functions are estimated using discrete data, the probability density functions will be approximated by (discrete) probability mass functions. It will be appreciated that there are other ways of estimating a probability mass function to those ways described above, and that for efficiency purposes simplified forms of this estimation can be performed.

Once the joint probability function has been determined in step 109, the method returns to step 101 and awaits the next predefined interval.

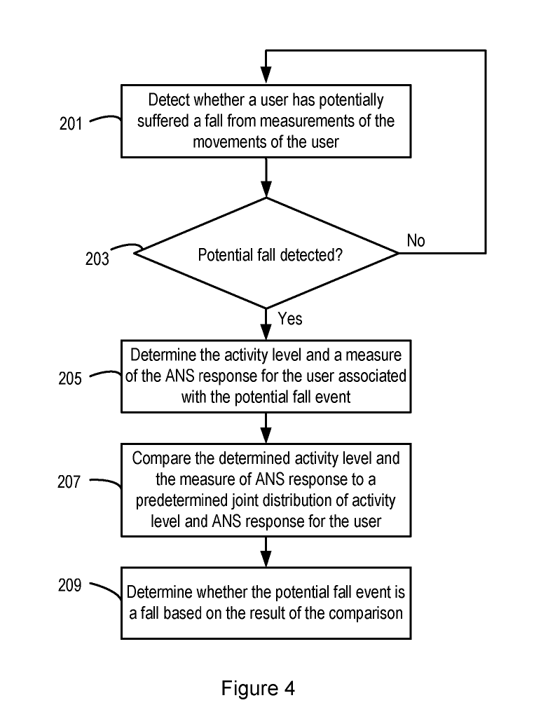

The flow chart in FIG. 4 illustrates a method of detecting a fall by a user according to the invention that makes use of the profile described above. In steps 201 and 203, the fall detector 2 determines whether the user may have suffered a fall from the measurements of the movement of the user.

The processor 8 in the fall detector 2 (or, in the alternative embodiment described above, the processor in the base station) determines if the user may have suffered a fall by extracting values for a feature or various features that are associated with a fall from the movement sensor measurements. Thus, in step 201, the accelerations and air pressure changes experienced by the fall detector 2 are measured using the accelerometer 4 and air pressure sensor 6, and these measurements are analysed by the processor 8 to determine whether the user might have suffered a fall.

A fall can be broadly characterised by, for example, a change in altitude of around 0.5 to 1.5 metres (the range may be different depending on the part of the body that the fall detector 2 is to be worn and the height of the user), culminating in a significant impact, followed by a period in which the user does not move very much. Thus, conventionally, in order to determine if a fall has taken place, the processor 8 needs to process the sensor measurements to extract values for features including a change in altitude (which is usually derived from the measurements from the pressure sensor 6, but can also or alternatively be derived from the measurements from the accelerometer 4, for example if the pressure sensor 6 is omitted), a maximum activity level (i.e. an impact) around the time that the change in altitude occurs (typically derived from the measurements from the accelerometer 4) and a period in which the user is relatively inactive following the impact (again typically derived from the measurements from the accelerometer 4). It will be appreciated that other features can further improve the detection algorithm. For example, the detection of a change in orientation upon falling can improve the likelihood that the signal is due to a fall.

A potential fall by the user can be identified where a subset or all of the above features are identified in the measurements. In other words, a potential fall may be identified where any one or more of the required height change, impact and inactivity period are detected in the measurements.

The analysis performed by the processor 8 in step 201 will not be described in further detail herein, but those skilled in the art will be aware of various algorithms and techniques that can be applied to determine whether a user may have suffered a fall from accelerometer and/or pressure sensor measurements.

If no potential fall has been detected in step 201 (i.e. no characteristics of a fall are evident from the measurements from the accelerometer 4 and/or pressure sensor 6, or insufficient characteristics of a fall are present in order for a potential fall to be detected), the method returns to step 201 and repeats on the next set of measurements.

If a potential fall has been detected in steps 201/203, the method moves to step 205 in which the processor 8 determines the activity level and a measure of the ANS response of the user (e.g. heart rate) that is associated with the potential fall event. The activity level and measure of the ANS response can be determined from measurements from the appropriate sensors (for example the accelerometer 4 and physiological characteristic sensor 16) that are collected shortly before and/or shortly after the potential fall event occurred. The length of time before and/or after the potential fall event for which measurements from the appropriate sensors are processed (and thus the amount of measurement data from those sensors that is to be processed) can depend on the power budget of the fall detector 2. In some embodiments, measurements in a 2-minute time window before the potential fall event and/or in a 2-minute time window after the potential fall event are processed.

The activity level and measure of the ANS response can be determined as described above with reference to step 101 of FIG. 3. Thus, an instantaneous activity level and measure of the ANS response can be determined or an average of the activity level and ANS response can be obtained for a predetermined time period. In some embodiments, the processor 8 can determine a measure of the change in activity level and ANS response across the event, i.e. the processor 8 can determine the activity level/heart rate measure before the event and the activity level/heart rate measure after the event, and calculate the difference.

In some embodiments, the physiological characteristic sensor 16 is only activated once a possible fall (or simply a change in altitude of at least 0.5 m detected in the measurements from the pressure sensor 6) has been detected from the analysis of the accelerometer 4 and/or pressure sensor 6 measurements, thus reducing the power consumption of the fall detector 2. As the analysis in step 201 is performed by the processor 8 substantially in real time or with only a small delay, the physiological characteristic sensor 16 will be activated shortly after a fall event has occurred. Where a separate sensor to the accelerometer 4 is used to determine the activity level, the separate sensor can be activated following the detection of a potential fall in the same way as the physiological characteristic sensor described above.

In alternative embodiments, the physiological characteristic sensor 16 may measure the physiological characteristic constantly or frequently whenever the fall detector 2 is in use (i.e. even when a possible fall has not yet been detected). This way, physiological characteristic measurements will be available to the processor 8 as soon as a possible fall is detected. Again, where a separate sensor to the accelerometer 4 is used to determine the activity level, the separate sensor can be activated constantly or frequently whenever the fall detector 2 is in use in the same way as the physiological characteristic sensor described above.

In step 207, the processor 8 compares the determined activity level and ANS response (e.g. heart rate) to the profile determined according to the method in FIG. 3 representing typical daily activities in order to determine whether the determined activity level and ANS response are consistent with that profile.

In some embodiments, step 207 can comprise using the profile to determine the likelihood of the user behaving with the determined activity level and ANS response. In these embodiments, a likelihood value p(xₑ|Θ) can be calculated where xₑ=[αₑ, rₑ]^{T} denotes the activity level and ANS response (e.g. heart rate) describing the potential fall event that are determined in step 205.

In other embodiments, the log-likelihood can be computed to simplify computations where a Gaussian model is used, for example.

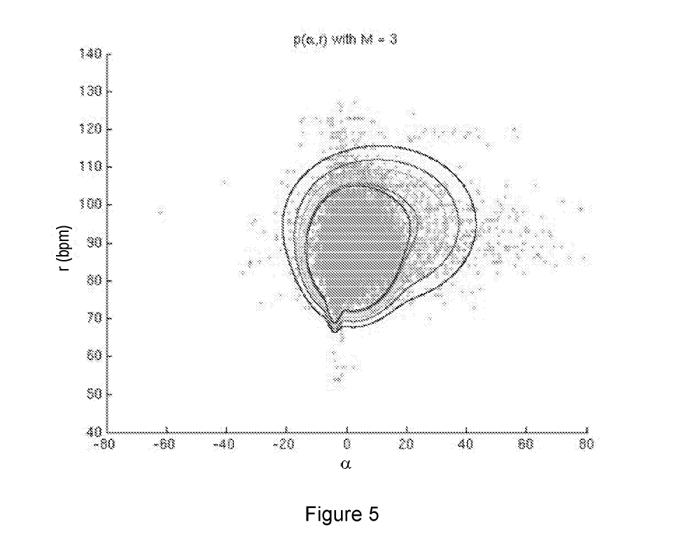

FIG. 5 is a contour plot of an exemplary joint distribution of heart rate and activity level (αᵢ, rᵢ) for a user using a Gaussian mixture model with M=3. In this example, the activity level was calculated using equation (1) above and the values averaged every 128 samples after removing the mean value. In general, data points (i.e. pairs of activity level and heart rate, (αᵢ, rᵢ)) that fall outside of the outermost contour curve have a low likelihood. The likelihood of the user behaving with a given activity level and ANS response further decreases with increasing distance of the data point from this curve.

In step 209, the processor 8 uses the result of step 207 to determine whether the potential fall detected in step 201 is an actual fall by the user.

If it was found in step 207 that the determined activity level and ANS response are consistent with the profile which represents the user's conditioning and typical daily activities, (e.g. the determined likelihood of the behaviour is high), then the behaviour is probably related to a daily activity by the user, and the potential fall event is classified as a ‘non-fall’.

However, if it was found in step 207 that the determined activity level and ANS response are not consistent with the profile (perhaps a low activity level with an unusual ANS response), e.g. the likelihood of the behaviour is low, then the behaviour is probably not related to a normal daily activity by the user and the potential fall event identified in step 201 is classified as an actual fall by the user.

In the embodiment of step 207 described above where the likelihood p(xₑ|Θ) is determined, the value of p(xₑ|Θ) will be quite low for a fall event as the physical fall can be traumatising and is often accompanied by little movement and a higher heart-rate. Therefore, in these embodiments, the value of the likelihood function p(xₑ|Θ) calculated in step 207 is compared to a threshold ηₜₕᵣₑₛ, to determine if the potential fall is an actual fall. If the value of p(xₑ|Θ) is less than the threshold ηₜₕᵣₑₛ then the potential fall event detected in step 201 can be classified as a fall. Otherwise, the potential fall event detected in step 201 is classified as a non-fall.

The value of ηₜₕᵣₑₛ is data-dependent and can be set according to the spread of the current probability density functions or the individual components of the mixture model in case a Gaussian mixture model is used, for example. The threshold value can also be based on prior data collected from a large number of users.

The threshold value can determine the overall performance and often involves a trade-off between the number of false-alarms and missed detections of real falls. It should be set to reach a given level of performance required for the application.

In another embodiment, the system can initially use a fixed threshold value and an initial probability density function estimate which is based on prior training data collected from a large number of users, or the threshold can be initially set using the user's personal data such as age, mobility, and overall cardiac health at time of subscription. The threshold can later be updated based on a fall rejection option where the user notifies the fall detector 2 if a detected fall (i.e. where an alarm is triggered) was actually a false alarm through a user input such as the use of another push button on the detector 2 or by holding down more than one button on the detector 2, for example. This way, a more personalized threshold can be set. The approach of using user feedback to correct the device might be less reliable in a system that does not use physiological data since certain movement features are often still not distinguishable between ADLs and actual falls. In that case, using such a user feedback approach might actually lead to an increase in missed real falls. In another embodiment, a computer in the call-centre with which the fall detector 2 is associated can send a signal to the fall detector 2 to adjust the threshold based on the activity level and heart-rate data collected during false alarms.

If the user is determined to have fallen, the processor 8 can trigger an alarm to obtain help for the user. After triggering the alarm, the process can return to step 201 to continue the monitoring of the user. If it is determined in step 209 that the user has not fallen, no alarm or alert will be triggered, and the process returns to step 201 to continue the monitoring of the user.

There is therefore provided a method for detecting falls and a fall detector that provides increased fall detection reliability compared to conventional techniques.

While the invention has been illustrated and described in detail in the drawings and foregoing description, such illustration and description are to be considered illustrative or exemplary and not restrictive; the invention is not limited to the disclosed embodiments.

Variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method of detecting a fall by a user, the method comprising:
detecting whether a user has potentially experienced a fall event from measurements of the movements of the user;
on detecting a potential fall event, determining the activity level of the user and a measure of a autonomic nervous system, ANS, response for the user associated with the potential fall event;
comparing the determined activity level and the measure of the ANS response to a user profile relating activity level and ANS response for the user; and
determining whether the potential fall event is a fall based on the result of the comparison.

2. The method as claimed in claim 1, wherein the step of comparing the determined activity level and the measure of the ANS response to a user profile comprises using the profile to determine the likelihood of the user having the determined activity level and measure of ANS response, and wherein the step of determining whether the potential fall event is a fall uses the determined likelihood.

3. The method as claimed in claim 2, wherein the user profile relates typical activity levels and typical ANS responses for the user, and wherein the step of determining whether the potential fall event is a fall comprises determining that the potential fall event is a fall if the determined likelihood is below a threshold, and determining that the potential fall event is not a fall if the determined likelihood is above a threshold.

4. The method as claimed in claim 2, wherein, in the event that it is determined that the potential fall event is a fall and an indication is subsequently received that the fall event was not a fall, the method further comprises the step of adjusting the value of the threshold.

5. The method as claimed in claim 4, wherein the indication that the fall event was not a fall is an input to the fall detector by the user or a signal received from a remote computer associated with the fall detector.

6. The method as claimed in claim 1, further comprising the step of:
determining the user profile relating activity level and ANS response for the user by:
(i) obtaining pairs of measurements of the activity level and ANS response for the user for a plurality of time periods; and
(ii) determining a joint distribution of activity level and ANS response for the user from the obtained pairs of measurements.

7. The method as claimed in claim 6, wherein the step of determining the user profile relating activity level and ANS response for the user comprises determining a plurality of user profiles relating activity level and ANS response for the user, wherein each profile relates the activity level and ANS response for a particular time period during the day.

8. The method as claimed in claim 6, wherein the step of obtaining pairs of measurements of the activity level and ANS response for the user comprises discarding any pair of measurements obtained for a time period in which a potential fall by the user is detected.

9. The method as claimed in claim 1, wherein the step of determining the activity level and a measure of an ANS response comprises determining the activity level and/or the measure of ANS response from the measurements of the movements of the user.

10. The method as claimed in claim 1, wherein the step of determining the activity level and a measure of the ANS response comprises determining the activity level from the measurements of the movements of the user and the measure of ANS response from measurements of a physiological characteristic of the user by a physiological characteristic sensor.

11. The method as claimed in claim 1, wherein the measure of ANS response is one or more of skin temperature, skin conductance, electromyography, heart rate and any other heart-related characteristic of the user.

12. The method as claimed in claim 1, wherein the step of detecting whether a user has potentially experienced a fall event comprises:
measuring the movements of the user; and
analyzing the measurements of the movements of the user to identify one or more characteristics associated with a fall.

13. The method as claimed in claim 12, wherein the one or more characteristics associated with a fall are selected from: (i) a height change, (ii) an impact, (iii) a free-fall, (iv) a change in orientation from upright to horizontal, and (v) a period of inactivity.

14. A computer program product comprising computer readable code embodied therein, the computer readable code being configured such that, upon execution by a suitable computer or processor, the computer or processor performs the method claimed claim 1.

15. A fall detector for detecting falls by a user, the fall detector comprising:
a movement sensor for measuring the movements of the user; and
a processor configured to:
detect whether the user has potentially experienced a fall event from measurements of the movements of the user from the sensor;
on detecting a potential fall event, determine the activity level of the user and a measure of a autonomic nervous system, ANS, response for the user associated with the potential fall event;
compare the determined activity level and the measure of the ANS response to a profile relating activity level and ANS response for the user; and
determine whether the potential fall event is a fall based on the result of the comparison.

16. The fall detector for detecting falls by a user according to claim 15 wherein the movement sensor is an accelerometer or an air pressure sensor.

17. The fall detector for detecting falls by a user according to claim 16 wherein the measure of an automatic nervous system response is in dependence of measurements of the accelerometer or air pressure sensor.

18. The fall detector for detecting falls by a user according to claim 15 wherein fall detector further comprises a skin conductivity sensor, a skin temperature sensor or a heart rate sensor for determining the measure of an automatic nervous system response.

19. The fall detector for detecting falls by a user according to claim 15 wherein the fall detector comprises a user interface enabling the user to notify the fall detector if the fall determined based on the result of the comparison was a false alarm.

20. The fall detector for detecting falls by a user according to claim 19 wherein a result of the comparison of the determined activity level and the measure of the ANS response to a profile relating activity level and ANS response for the user is dependent on a threshold, the processor being configured to adjust the threshold in dependence of the user notifying the fall detector that the determined fall was a false alarm.
